(19) United States
(12) Patent Application Publication
(10) Pub. No.: US 2018/0016277 A1
(43) Pub. Date: Jan. 18, 2018

(54) DEUTERATED TIC10

(71) Applicant: Concert Pharmaceuticals, Inc., Lexington, MA (US)

(72) Inventor: I. Robert Silverman, Arlington, MA (US)

(21) Appl. No.: 15/547,040

(22) PCT Filed: Jan. 27, 2016
(86) PCT No.: PCT/US2016/015063

§ 371 Date: Jul. 27, 2017

Related U.S. Application Data
(60) Provisional application No. 62/108,133, filed on Jan. 27, 2015.

(51) Int. Cl. C07D 471/14; C07B 59/00

(52) U.S. Cl. CPC C07D 471/14; C07B 59/002; C07B 2200/05

(57) ABSTRACT

This invention relates to novel imidazo[1,2-a]pyrido[3,4-e]pyrimidin-5(1H)-one compounds, and pharmaceutically acceptable salts thereof. This invention also provides compositions comprising a compound of this invention and the use of such compositions alone or in combination with other therapeutics in the treatment of diseases and conditions that are beneficially treated by administering an inducer of the gene encoding tumor necrosis factor (TNF) related apoptosis-inducing ligand (TRAIL) superfamily member 10.

### CROSS REFERENCE TO RELATED APPLICATIONS

This application claims the benefit of U.S. Provisional Ser. No. 62/108,133 filed Jan. 27, 2015. This disclosure of the prior application is considered part of (and is incorporated by reference in) the disclosure of this application.

### BACKGROUND OF THE INVENTION

Many current medicines suffer from poor absorption, distribution, metabolism and/or excretion (ADME) properties that prevent their wider use or limit their use in certain indications. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment. A rapidly metabolized drug may also expose patients to undesirable toxic or reactive metabolites.

Another ADME limitation that affects many medicines is the formation of toxic or biologically reactive metabolites. As a result, some patients receiving the drug may experience toxicities, or the safe dosing of such drugs may be limited such that patients receive a suboptimal amount of the active agent. In certain cases, modifying dosing intervals or formulation approaches can help to reduce clinical adverse effects, but often the formation of such undesirable metabolites is intrinsic to the metabolism of the compound.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

### SUMMARY OF THE INVENTION

This invention relates to novel imidazo[1,2-a]pyrido[3,4-e]pyrimidin-5(1H)-one compounds, and pharmaceutically acceptable salts thereof. This invention also provides compositions comprising a compound of this invention and the use of such compositions alone or in combination with other therapeutics in the treatment of diseases and conditions that are beneficially treated by administering an inducer of the gene encoding tumor necrosis factor (TNF) related apoptosis-inducing ligand (TRAIL) superfamily member 10.

TIC10 is also known as NSC-350625 and ONC-201, and by the chemical name, 7-Benzyl-4-(2-methylbenzyl)-2,4,6,7,8,9-hexahydroimidazo[1,2-a]pyrido[3,4-e]pyrimidin-5(1H)-one. TIC10 inactivates kinases Akt and extracellular signal-regulated kinase (ERK), leading to the translocation of Foxo3a into the nucleus, where it binds to the TRAIL promoter to up-regulate gene transcription, resulting in apoptosis in cancer cells.

TIC10 is currently beginning human clinical trials in cancer, as an IND was recently filed.

Despite the potential beneficial activities of TIC10, there is a continuing need for new compounds to treat cancer.

### DETAILED DESCRIPTION OF THE INVENTION

### Definitions

The term “treat” means decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of TIC10 will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species in which the chemical structure differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert” and “t-” each refer to tertiary. “US” refers to the United States of America.

“Substituted with deuterium” refers to the replacement of one or more hydrogen atoms with a corresponding number of deuterium atoms.

Throughout this specification, a variable may be referred to generally (e.g., “each R”) or may be referred to specifically (e.g., R¹, R², R³, etc.). Unless otherwise indicated, when a variabR¹ is referred to generally, it is meant to include all specific embodiments of that particular variable.

### Therapeutic Compounds

The present invention provides a compound of Formula I:

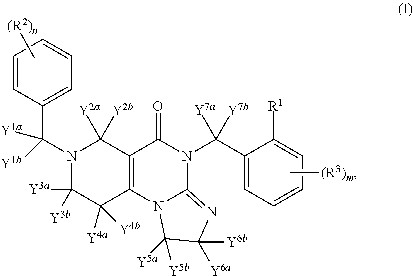

or a pharmaceutically acceptable salt thereof, wherein:
each of Y^{1a}, Y^{1b}, Y^{2a}, Y^{2b}, Y^{3a}, Y^{3b}, Y^{4a}, Y^{4b}, Y^{5a}, Y^{5b}, Y^{6a}, Y^{6b}, Y^{7a}, and Y^{7b} are independently selected from hydrogen and deuterium;

R¹ is selected from —CH₃, —CH₂D, —CHD₂ and —CD₃;

each R², when present, is deuterium;

each R³, when present, is deuterium;

n is 0, 1, 2, 3, 4, or 5;

m is 0, 1, 2, 3, or 4; and

when R¹ is —CH₃, n is 0 and m is 0, then at least one of Y^{1a}, Y^{1b}, Y^{2a}, Y^{2b}, Y^{3a}, Y^{3b}, Y^{4a}, Y^{4b}, Y^{5a}, Y^{5b}, Y^{6a}, Y^{6b}, Y^{7a}, and Y^{7b} is deuterium.

In one embodiment of a compound of Formula I, the invention provides a compound of Formula Ia:

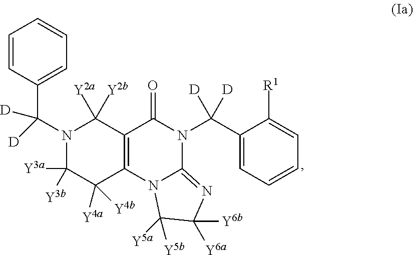

or a pharmaceutically acceptable salt thereof, wherein: each of Y^{2a}, Y^{2b}, Y^{3a}, Y^{3b}, Y^{4a}, Y^{4b}, Y^{5a}, Y^{5b}, Y^{6a}, and Y^{6b} are independently selected from hydrogen and deuterium; and

R¹ is selected from —CH₃, and —CD₃.

In one embodiment of a compound of Formula I, the invention provides a compound of Formula Ib:

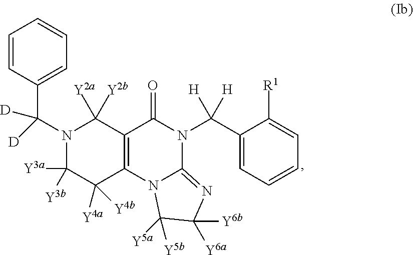

or a pharmaceutically acceptable salt thereof, wherein:

each of Y^{2a}, Y^{2b}, Y^{3a}, Y^{3b}, Y^{4a}, Y^{4b}, Y^{5a}, Y^{5b}, Y^{6a}, and Y^{6b} are independently selected from hydrogen and deuterium; and

R¹ is selected from —CH₃, and —CD₃.

In one embodiment of a compound of Formula I, the invention provides a compound of Formula Ic:

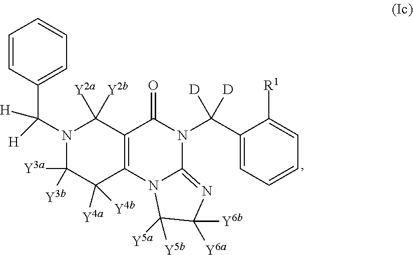

or a pharmaceutically acceptable salt thereof, wherein:

each of Y^{2a}, Y^{2b}, Y^{3a}, Y^{3b}, Y^{4a}, Y^{4b}, Y^{5a}, Y^{5b}, Y^{6a}, and Y^{6b} are independently selected from hydrogen and deuterium; and

R¹ is selected from —CH₃, and —CD₃.

In certain embodiments of a compound of Formula I, Y^{1a} and Y^{1b} are the same; Y^{2a} and Y^{2b} are the same; Y^{3a} and Y^{3b} are the same; Y^{4a} and Y^{4b} are the same; Y^{5a} and Y^{5b} are the same; Y^{6a} and Y^{6b} are the same; and Y^{7a} and Y^{7b} are the same. In one aspect of these embodiments, Y^{1a} and Y^{1b} are hydrogen. In an alternate aspect of these embodiments, Y^{1a} and Y^{1b} are deuterium. In another aspect of these embodiments, Y^{2a} and Y^{2b} are hydrogen. In an alternate aspect of these embodiments, Y^{2a} and Y^{2b} are deuterium. In yet another aspect of these embodiments, Y^{3a} and Y^{3b} are hydrogen. In an alternate aspect of these embodiments, Y^{3a} and Y^{3b} are deuterium. In still another aspect of these embodiments, Y^{4a} and Y^{4b} are hydrogen. In an alternate aspect of these embodiments, Y^{4a} and Y^{4b} are deuterium. In another aspect of these embodiments, Y^{5a} and Y^{5b} are hydrogen. In an alternate aspect of these embodiments, Y^{5a} and Y^{5b} are deuterium. In another aspect of these embodiments, Y^{6a} and Y^{6b} are hydrogen. In an alternate aspect of these embodiments, Y^{6a} and Y^{6b} are deuterium. In still another aspect of these embodiments, Y^{7a} and Y^{7b} are hydrogen. In an alternate aspect of these embodiments, Y^{7a} and Y^{7b} are deuterium.

In certain embodiments of a compound Formula I, R¹ is selected from —CH₃ and —CD₃. In one aspect of these embodiments, R¹ is —CH₃. In an alternate aspect of these embodiments R¹ is —CD₃.

In certain more specific embodiments of a compound Formula I, Y^{1a} and Y^{1b} are the same; Y^{2a}, Y^{2b}, Y^{3a}, Y^{3b}, Y^{4a} and Y^{4b} are the same; Y^{5a}, Y^{5b}, Y^{6a} and Y^{6b} are the same; and Y^{7a} and Y^{7b} are the same. In one aspect of these embodiments, Y^{1a} and Y^{1b} are the same; Y^{2a}, Y^{2b}, Y^{3a}, Y^{3b}, Y^{4a} and Y^{4b} are deuterium; Y^{5a}, Y^{5b}, Y^{6a} and Y^{6b} are deuterium; and Y^{7a} and Y^{7b} are the same. In another aspect of these embodiments, Y^{1a} and Y^{1b} are the same; Y^{2a}, Y^{2b}, Y^{3a}, Y^{3b}, Y^{4a} and Y^{4b} are hydrogen; Y^{5a}, Y^{5b}, Y^{6a} and Y^{6b} are deuterium; and Y^{7a} and Y^{7b} are the same. In still another aspect of these embodiments, Y^{1a} and Y^{1b} are the same; Y^{2a}, Y^{2b}, Y^{3a}, Y^{3b}, Y^{4a} and Y^{4b} are deuterium; Y^{5a}, Y^{5b}, Y^{6a} and Y^{6b} are hydrogen; and Y^{7a} and Y^{7b} are the same. In yet another aspect of these embodiments, Y^{1a} and Y^{1b} are the same; Y^{2a}, Y^{2b}, Y^{3a}, Y^{3b}, Y^{4a} and Y^{4b} are hydrogen; Y^{5a}, Y^{5b}, Y^{6a} and Y^{6b} are hydrogen; and Y^{7a} and Y^{7b} are the same.

In certain embodiments of a compound of Formula I, n is 0 or 5. In one aspect of these embodiments, n is 0. In an alternate aspect of these embodiments, n is 5.

In certain embodiments of a compound of Formula I, m is 0 or 4. In one aspect of these embodiments, m is 0. In an alternate aspect of these embodiments, m is 4.

In certain embodiments of a compound of Formula Ia, Ib or Ic, Y^{2a} and Y^{2b} are the same; Y^{3a} and Y^{3b} are the same; Y^{4a} and Y^{4b} are the same; Y^{5a} and Y^{5b} are the same; and Y^{6a} and Y^{6b} are the same. In one aspect of these embodiments, Y^{2a} and Y^{2b} are hydrogen. In an alternate aspect of these embodiments, Y^{2a} and Y^{2b} are deuterium. In another aspect of these embodiments, Y^{3a} and Y^{3b} are hydrogen. In an alternate aspect of these embodiments, Y^{3a} and Y^{3b} are deuterium. In yet another aspect of these embodiments, Y^{4a} and Y^{4b} are hydrogen. In an alternate aspect of these embodiments, Y^{4a} and Y^{4b} are deuterium. In still another aspect of these embodiments, Y^{5a} and Y^{5b} are hydrogen. In an alternate aspect of these embodiments, Y^{5a} and Y^{5b} are deuterium. In another aspect of these embodiments, Y^{6a} and Y^{6b} are hydrogen. In an alternate aspect of these embodiments, Y^{6a} and Y^{6b} are deuterium.

In certain more specific embodiments of a compound Formula Ia, Ib or Ic, Y^{1a} and Y^{1b} are the same; Y^{2a}, Y^{2b}, Y^{3a}, Y^{3b}, Y^{4a} and Y^{4b} are the same; Y^{5a}, Y^{5b}, Y^{6a} and Y^{6b} are the same; and Y^{7a} and Y^{7b} are the same. In one aspect of these embodiments, Y^{1a} and Y^{1b} are the same; Y^{2a}, Y^{2b}, Y^{3a}, Y^{3b}, Y^{4a} and Y^{4b} are deuterium; Y^{5a}, Y^{5b}, Y^{6a} and Y^{6b} are deuterium; and Y^{7a} and Y^{7b} are the same. In another aspect of these embodiments, Y^{1a} and Y^{1b} are the same; Y^{2a}, Y^{2b}, Y^{3a}, Y^{3b}, Y^{4a} and Y^{4b} are hydrogen; Y^{5a}, Y^{5b}, Y^{6a} and Y^{6b} are deuterium; and Y^{7a} and Y^{7b} are the same. In still another aspect of these embodiments, Y^{1a} and Y^{1b} are the same; Y^{2a}, Y^{2b}, Y^{3a}, Y^{3b}, Y^{4a} and Y^{4b} are deuterium; Y^{5a} Y^{5b}, Y^{6a} and Y^{6b} are hydrogen; and Y^{7a} and Y^{7b} are the same. In yet another aspect of these embodiments, Y^{1a} and Y^{1b} are the same; Y^{2a}, Y^{2b}, Y^{3a}, Y^{3b}, Y^{4a} and Y^{4b} are hydrogen; Y^{5a}, Y^{5b}, Y^{6a} and Y^{6b} are hydrogen; and Y^{7a} and Y^{2b} are the same.

In one embodiment, the compound of Formula I is not a compound wherein each of Y^{1a}, Y^{1b}, Y^{2a} Y^{2b}, Y^{3a}, Y^{3b}, Y^{4a}, Y⁴b, Y^{5a} Y^{5b}, Y^{6a}, Y^{6b}, Y^{7a}, and Y^{7b} is deuterium; R¹ is —CD₃; n is 5; each R² is deuterium; m is 4; and each R³ is deuterium.

In one embodiment of a compound of Formula I, R¹ is —CD₃; n is 0; m is 0; Y^{1a} and Y^{1b} are each hydrogen; Y^{2a} and Y^{2b} are the same; Y^{3a} and Y^{3b} are the same; Y^{4a} and Y^{4b} are the same; Y^{5a} and Y^{5b} are the same; Y^{6a} and Y^{6b} are the same; Y^{7a} and Y^{7b} are each hydrogen; and the compound is selected from any one of the compounds (Cmpd) set forth in Table 1a (below):

TABLE 1a
Exemplary Embodiments of Formula I, where R¹ is —CD₃

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 100a | H | H | H | H | H |
| 101a | D | H | H | H | H |
| 102a | H | D | H | H | H |
| 103a | H | H | D | H | H |
| 104a | H | H | H | D | H |
| 105a | H | H | H | H | D |
| 106a | D | D | H | H | H |
| 107a | D | H | D | H | H |
| 108a | D | H | H | D | H |
| 109a | D | H | H | H | D |
| 110a | H | D | D | H | H |
| 111a | H | D | H | D | H |
| 112a | H | D | H | H | D |
| 113a | H | H | D | D | H |
| 114a | H | H | D | H | D |
| 115a | H | H | H | D | D |
| 116a | D | D | D | H | H |
| 117a | D | D | H | D | H |
| 118a | D | D | H | H | D |
| 119a | D | H | D | D | H |
| 120a | D | H | D | H | D |
| 121a | D | H | H | D | D |
| 122a | H | D | D | D | H |
| 123a | H | D | D | H | D |
| 124a | H | D | H | D | D |
| 125a | H | H | D | D | D |
| 126a | D | D | D | D | H |
| 127a | D | D | D | H | D |
| 128a | D | D | H | D | D |
| 129a | D | H | D | D | D |
| 130a | H | D | D | D | D |
| 131a | D | D | D | D | D |

or a pharmaceutically acceptable salt thereof.

In one embodiment of a compound of Formula I, R¹ is —CH₃; n is 0; m is 0; Y^{1a} and Y^{1b} are each hydrogen; Y^{2a} and Y^{2b} are the same; Y^{3a} and Y^{3b} are the same; Y^{4a} and Y^{4b} are the same; Y^{5a} and Y^{5b} are the same; Y^{6a} and Y^{6b} are the same; Y^{7a} and Y^{7b} are each hydrogen; and the compound is selected from any one of the compounds (Cmpd) set forth in Table 1b (below):

TABLE 1b
Exemplary Embodiments of Formula I, where R¹ is —CH₃

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 101b | D | H | H | H | H |
| 102b | H | D | H | H | H |
| 103b | H | H | D | H | H |
| 104b | H | H | H | D | H |
| 105b | H | H | H | H | D |
| 106b | D | D | H | H | H |
| 107b | D | H | D | H | H |
| 108b | D | H | H | D | H |
| 109b | D | H | H | H | D |
| 110b | H | D | D | H | H |
| 111b | H | D | H | D | H |
| 112b | H | D | H | H | D |
| 113b | H | H | D | D | H |
| 114b | H | H | D | H | D |
| 115b | H | H | H | D | D |
| 116b | D | D | D | H | H |
| 117b | D | D | H | D | H |
| 118b | D | D | H | H | D |
| 119b | D | H | D | D | H |
| 120b | D | H | D | H | D |
| 121b | D | H | H | D | D |
| 122b | H | D | D | D | H |
| 123b | H | D | D | H | D |
| 124b | H | D | H | D | D |
| 125b | H | H | D | D | D |
| 126b | D | D | D | D | H |
| 127b | D | D | D | H | D |
| 128b | D | D | H | D | D |
| 129b | D | H | D | D | D |
| 130b | H | D | D | D | D |
| 131b | D | D | D | D | D |

or a pharmaceutically acceptable salt thereof.

In one embodiment of a compound of Formula Ia, R¹ is —CD₃; Y^{2a} and Y^{2b} are the same; Y^{3a} and Y^{3b} are the same; Y^{4a} and Y^{4b} are the same; Y^{5a} and Y^{5b} are the same; Y^{6a} and Y^{6b} are the same; and the compound is selected from any one of the compounds (Cmpd) set forth in Table 2a (below):

TABLE 2a
Exemplary Embodiments of Formula Ia, where R¹ is —CD₃

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 200a | H | H | H | H | H |
| 201a | D | H | H | H | H |
| 202a | H | D | H | H | H |
| 203a | H | H | D | H | H |
| 204a | H | H | H | D | H |
| 205a | H | H | H | H | D |
| 206a | D | D | H | H | H |
| 207a | D | H | D | H | H |
| 208a | D | H | H | D | H |
| 209a | D | H | H | H | D |
| 210a | H | D | D | H | H |
| 211a | H | D | H | D | H |
| 212a | H | D | H | H | D |
| 213a | H | H | D | D | H |
| 214a | H | H | D | H | D |
| 215a | H | H | H | D | D |
| 216a | D | D | D | H | H |
| 217a | D | D | H | D | H |
| 218a | D | D | H | H | D |
| 219a | D | H | D | D | H |
| 220a | D | H | D | H | D |
| 221a | D | H | H | D | D |
| 222a | H | D | D | D | H |
| 223a | H | D | D | H | D |
| 224a | H | D | H | D | D |
| 225a | H | H | D | D | D |
| 226a | D | D | D | D | H |
| 227a | D | D | D | H | D |
| 228a | D | D | H | D | D |
| 229a | D | H | D | D | D |
| 230a | H | D | D | D | D |
| 231a | D | D | D | D | D |

or a pharmaceutically acceptable salt thereof, wherein any atom not designated as deuterium is present at its natural isotopic abundance.

In one embodiment of a compound of Formula Ia, R¹ is —CH₃; Y^{2a} and Y^{2b} are the same; Y^{3a} and Y^{3b} are the same; Y^{4a} and Y^{4b} are the same; Y^{5a} and Y^{5b} are the same; Y^{6a} and Y^{6b} are the same; and the compound is selected from any one of the compounds (Cmpd) set forth in Table 2b (below):

TABLE 2b
Exemplary Embodiments of Formula Ia, where R¹ is —CH₃

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 200b | H | H | H | H | H |
| 201b | D | H | H | H | H |
| 202b | H | D | H | H | H |
| 203b | H | H | D | H | H |
| 204b | H | H | H | D | H |
| 205b | H | H | H | H | D |
| 206b | D | D | H | H | H |
| 207b | D | H | D | H | H |
| 208b | D | H | H | D | H |
| 209b | D | H | H | H | D |
| 210b | H | D | D | H | H |
| 211b | H | D | H | D | H |
| 212b | H | D | H | H | D |
| 213b | H | H | D | D | H |
| 214b | H | H | D | H | D |
| 215b | H | H | H | D | D |
| 216b | D | D | D | H | H |
| 217b | D | D | H | D | H |
| 218b | D | D | H | H | D |
| 219b | D | H | D | D | H |
| 220b | D | H | D | H | D |
| 221b | D | H | H | D | D |
| 222b | H | D | D | D | H |
| 223b | H | D | D | H | D |
| 224b | H | D | H | D | D |
| 225b | H | H | D | D | D |
| 226b | D | D | D | D | H |
| 227b | D | D | D | H | D |
| 228b | D | D | H | D | D |
| 229b | D | H | D | D | D |
| 230b | H | D | D | D | D |
| 231b | D | D | D | D | D |

or a pharmaceutically acceptable salt thereof, wherein any atom not designated as deuterium is present at its natural isotopic abundance.

In one embodiment of a compound of Formula Ib, R¹ is —CD₃; Y^{2a} and Y^{2b} are the same; Y^{3a} and Y^{3b} are the same; Y^{4a} and Y^{4b} are the same; Y^{5a} and Y^{5b} are the same; Y^{6a} and Y^{6b} are the same; and the compound is selected from any one of the compounds (Cmpd) set forth in Table 3a (below):

TABLE 3a
Exemplary Embodiments of Formula Ib, where R¹ is —CD₃

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 300a | H | H | H | H | H |
| 301a | D | H | H | H | H |
| 302a | H | D | H | H | H |
| 303a | H | H | D | H | H |
| 304a | H | H | H | D | H |
| 305a | H | H | H | H | D |
| 306a | D | D | H | H | H |
| 307a | D | H | D | H | H |
| 308a | D | H | H | D | H |
| 309a | D | H | H | H | D |
| 310a | H | D | D | H | H |
| 311a | H | D | H | D | H |
| 312a | H | D | H | H | D |
| 313a | H | H | D | D | H |
| 314a | H | H | D | H | D |
| 315a | H | H | H | D | D |
| 316a | D | D | D | H | H |
| 317a | D | D | H | D | H |
| 318a | D | D | H | H | D |
| 319a | D | H | D | D | H |
| 320a | D | H | D | H | D |
| 321a | D | H | H | D | D |
| 322a | H | D | D | D | H |
| 323a | H | D | D | H | D |
| 324a | H | D | H | D | D |
| 325a | H | H | D | D | D |
| 326a | D | D | D | D | H |
| 327a | D | D | D | H | D |
| 328a | D | D | H | D | D |
| 329a | D | H | D | D | D |
| 330a | H | D | D | D | D |
| 331a | D | D | D | D | D |

or a pharmaceutically acceptable salt thereof, wherein any atom not designated as deuterium is present at its natural isotopic abundance.

In one embodiment of a compound of Formula Ib, R¹ is —CH₃; Y^{2a} and Y^{2b} are the same; Y^{3a} and Y^{3b} are the same; Y^{4a} and Y^{4b} are the same; Y^{5a} and Y^{5b} are the same; Y^{6a} and Y^{6b} are the same; and the compound is selected from any one of the compounds (Cmpd) set forth in Table 3b (below):

TABLE 3b
Exemplary Embodiments of Formula Ib, where R¹ is —CH₃

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 300b | H | H | H | H | H |
| 301b | D | H | H | H | H |
| 302b | H | D | H | H | H |
| 303b | H | H | D | H | H |
| 304b | H | H | H | D | H |
| 305b | H | H | H | H | D |
| 306b | D | D | H | H | H |
| 307b | D | H | D | H | H |
| 308b | D | H | H | D | H |
| 309b | D | H | H | H | D |
| 310b | H | D | D | H | H |
| 311b | H | D | H | D | H |
| 312b | H | D | H | H | D |
| 313b | H | H | D | D | H |
| 314b | H | H | D | H | D |
| 315b | H | H | H | D | D |
| 316b | D | D | D | H | H |
| 317b | D | D | H | D | H |
| 318b | D | D | H | H | D |
| 319b | D | H | D | D | H |
| 320b | D | H | D | H | D |
| 321b | D | H | H | D | D |
| 322b | H | D | D | D | H |
| 323b | H | D | D | H | D |
| 324b | H | D | H | D | D |
| 325b | H | H | D | D | D |
| 326b | D | D | D | D | H |
| 327b | D | D | D | H | D |
| 328b | D | D | H | D | D |
| 329b | D | H | D | D | D |
| 330b | H | D | D | D | D |
| 331b | D | D | D | D | D |

or a pharmaceutically acceptable salt thereof, wherein any atom not designated as deuterium is present at its natural isotopic abundance.

In one embodiment of a compound of Formula Ic, R¹ is —CD₃; Y^{2a} and Y^{2b} are the same; Y^{3a} and Y^{3b} are the same; Y^{4a} and Y^{4b} are the same; Y^{5a} and Y^{5b} are the same; Y^{6a} and Y^{6b} are the same; and the compound is selected from any one of the compounds (Cmpd) set forth in Table 4a (below):

TABLE 4a
Exemplary Embodiments of Formula Ic, where R¹ is —CD₃

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 400a | H | H | H | H | H |
| 401a | D | H | H | H | H |
| 402a | H | D | H | H | H |
| 403a | H | H | D | H | H |
| 404a | H | H | H | D | H |
| 405a | H | H | H | H | D |
| 406a | D | D | H | H | H |
| 407a | D | H | D | H | H |
| 408a | D | H | H | D | H |
| 409a | D | H | H | H | D |
| 410a | H | D | D | H | H |
| 411a | H | D | H | D | H |
| 412a | H | D | H | H | D |
| 413a | H | H | D | D | H |
| 414a | H | H | D | H | D |
| 415a | H | H | H | D | D |
| 416a | D | D | D | H | H |
| 417a | D | D | H | D | H |
| 418a | D | D | H | H | D |
| 419a | D | H | D | D | H |
| 420a | D | H | D | H | D |
| 421a | D | H | H | D | D |
| 422a | H | D | D | D | H |
| 423a | H | D | D | H | D |
| 424a | H | D | H | D | D |
| 425a | H | H | D | D | D |
| 426a | D | D | D | D | H |
| 427a | D | D | D | H | D |
| 428a | D | D | H | D | D |
| 429a | D | H | D | D | D |
| 430a | H | D | D | D | D |
| 431a | D | D | D | D | D |

or a pharmaceutically acceptable salt thereof, wherein any atom not designated as deuterium is present at its natural isotopic abundance.

In one embodiment of a compound of Formula Ic, R¹ is —CH₃; Y^{2a} and Y^{2b} are the same; Y^{3a} and Y^{3b} are the same; Y^{4a} and Y^{4b} are the same; Y^{5a} and Y^{5b} are the same; Y^{6a} and Y^{6b} are the same; and the compound is selected from any one of the compounds (Cmpd) set forth in Table 4b (below):

TABLE 4b
Exemplary Embodiments of Formula Ic, where R¹ is —CH₃

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 400b | H | H | H | H | H |
| 401b | D | H | H | H | H |
| 402b | H | D | H | H | H |
| 403b | H | H | D | H | H |
| 404b | H | H | H | D | H |
| 405b | H | H | H | H | D |
| 406b | D | D | H | H | H |
| 407b | D | H | D | H | H |
| 408b | D | H | H | D | H |
| 409b | D | H | H | H | D |
| 410b | H | D | D | H | H |
| 411b | H | D | H | D | H |
| 412b | H | D | H | H | D |
| 413b | H | H | D | D | H |
| 414b | H | H | D | H | D |
| 415b | H | H | H | D | D |
| 416b | D | D | D | H | H |
| 417b | D | D | H | D | H |
| 418b | D | D | H | H | D |
| 419b | D | H | D | D | H |
| 420b | D | H | D | H | D |
| 421b | D | H | H | D | D |
| 422b | H | D | D | D | H |
| 423b | H | D | D | H | D |
| 424b | H | D | H | D | D |
| 425b | H | H | D | D | D |
| 426b | D | D | D | D | H |
| 427b | D | D | D | H | D |
| 428b | D | D | H | D | D |
| 429b | D | H | D | D | D |
| 430b | H | D | D | D | D |
| 431b | D | D | D | D | D |

or a pharmaceutically acceptable salt thereof, wherein any atom not designated as deuterium is present at its natural isotopic abundance.

In one embodiment of a compound of Formula Ib, the compound is selected from

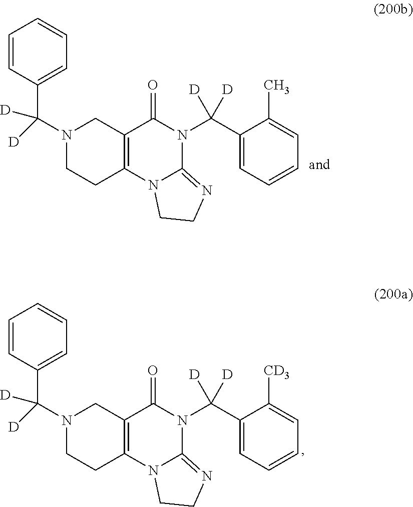 [SMILES: [2H]C([2H])(c1ccccc1)N1CCC2=C(C1)C(=O)N(C([2H])([2H])c1ccccc1C)C1=NCCN12 ; [2H]C([2H])([2H])c1ccccc1C([2H])([2H])N1C(=O)C2=C(CCN(C([2H])([2H])c3ccccc3)C2)N2CCN=C21]

or a pharmaceutically acceptable salt thereof, wherein any atom not specifically designated as deuterium is present at its natural abundance.

In one embodiment of a compound of Formula Ib, the compound is selected from

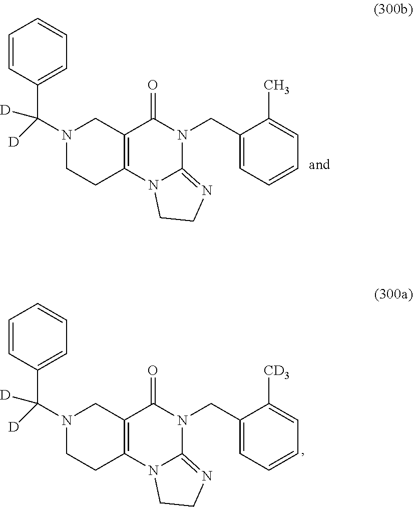 [SMILES: [2H]C([2H])(c1ccccc1)N1CCC2=C(C1)C(=O)N(Cc1ccccc1C)C1=NCCN12 ; [2H]C([2H])([2H])c1ccccc1CN1C(=O)C2=C(CCN(C([2H])([2H])c3ccccc3)C2)N2CCN=C12]

or a pharmaceutically acceptable salt thereof, wherein any atom not specifically designated as deuterium is present at its natural abundance.

In one embodiment of a compound of Formula Ic, the compound is selected from

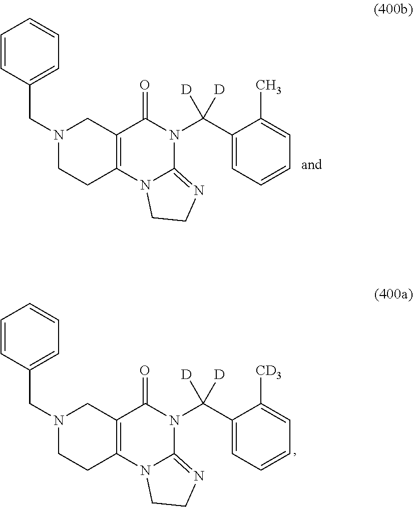 [SMILES: [2H]C([2H])(c1ccccc1C)N1C(=O)C2=C(CCN(Cc3ccccc3)C2)N2CCN=C21 ; [2H]C([2H])([2H])c1ccccc1C([2H])([2H])N1C(=O)C2=C(CCN(Cc3ccccc3)C2)N2CCN=C21]

or a pharmaceutically acceptable salt thereof, wherein any atom not specifically designated as deuterium is present at its natural abundance.

In another set of embodiments, any atom not designated as deuterium in any of the embodiments set forth above is present at its natural isotopic abundance.

The synthesis of compounds of Formula I, Ia, Ib and Ic may be readily achieved by synthetic chemists of ordinary skill by reference to the Exemplary Synthesis and Examples disclosed herein. Relevant procedures analogous to those of use for the preparation of compounds of Formula I, Ia, Ib, Ic and intermediates thereof are disclosed, for instance in Jacob, N. T.; et al., Angew Chem Int Ed 2014, 53(26): 6628 and US Patent publication 2014/0335048.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Synthesis

A convenient method for synthesizing compounds of Formula I is depicted in Scheme 1, below. Also, a compound of Formula Ia, Ib or Ic may be prepared according to Scheme 1.

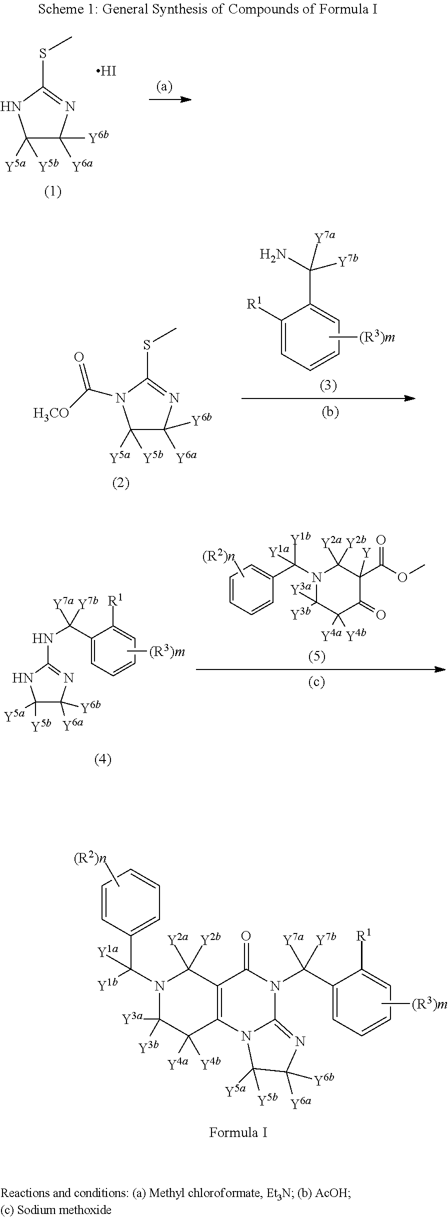

In a manner analogous to the procedure described in US20140335048 and Jacob, N. et al., Angew Chem Int Ed 2014, 53(26): 6628, appropriately deuterated mercapto-imidazoline (1) is treated with methyl chloroformate under basic reaction conditions to produce protected deuterated mercapto-imidazoline intermediate (2), which is condensed with appropriately deuterated arylamine intermediate (3) in the presence of acetic acid to afford appropriately deuterated amino-imidazoline intermediate (4). Subsequent cyclo-condensation of intermediate (4) with appropriately deuterated keto ester intermediate (5), wherein Y is hydrogen or deuterium, in the presence of sodium methoxide under reflux conditions furnishes correspondingly deuterated compounds of Formula I.

Using commercially available reagents and solvents and deuterated reagents that can be readily prepared by known methods, compounds of Formula I can be prepared with greater than 90% or greater than 95% deuterium incorporation at each position designated as D (see below for details).

Appropriately deuterated intermediate (1), for use in the preparation of compounds of Formula I according to Scheme 1 are commercially available or may be prepared from corresponding deuterated reagents depicted below.

Intermediate (1a), 2-methylthio-2-imidazoline-4,5-d₄, hydroiodide (99 atom % D) is commercially available or is prepared according to a method described in WO 2003056299. Intermediate (Ib) can be prepared as outlined in Scheme 2 below.

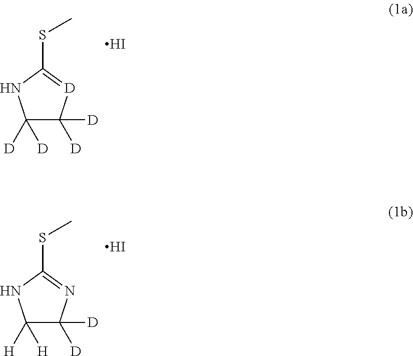

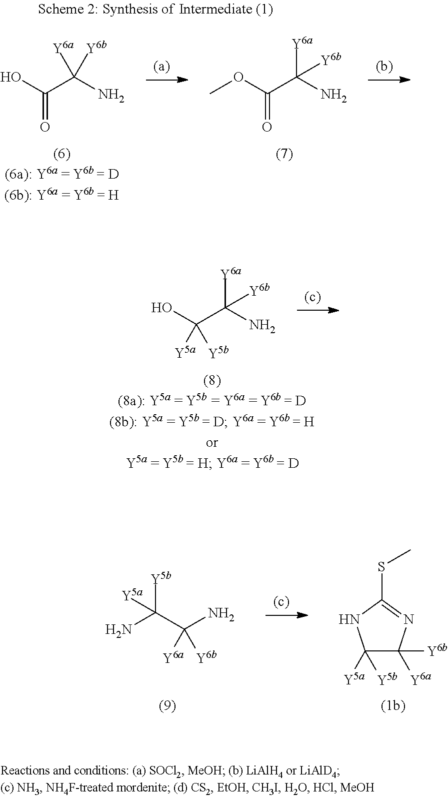

For instance, commercially available glycine-2,2-d₂ (98 atom % D) (6a) is esterified employing methods known in the art to furnish intermediate (7), which is reduced with LiAlH₄ or LiAlD₄ in accordance with a procedure described by Tan, P-Z., et al., Journal of Labelled Compounds & Radiopharmaceuticals (1999), 42(5), 457-467, to provide appropriately deuterated intermediate (8). Intermediate (8) is subsequently treated with an amine reagent such as NH₃ in the presence of fluorinated zeolites to furnish appropriately deuterated intermediate (9) in a manner analogous to a method described in JP 05271159 A. Finally, appropriate intermediate (9) is treated with carbon disulfide and iodomethane at elevated temperature to furnish appropriately deuterated intermediate (1b)/(1c), following a method described in WO 2003056299. The use of commercially available (6a) can thus afford compounds of Formula I wherein each Y⁶ has between 95-98% deuterium incorporation. Using LiAlD₄ to prepare intermediate 8, compounds of Formula I can be prepared wherein each Y⁵ has at least 98% deuterium incorporation.

Appropriately deuterated intermediate (3), for use in the preparation of compounds of Formula I according to Scheme 1, may be prepared from corresponding deuterated reagents exemplified in Scheme 3.

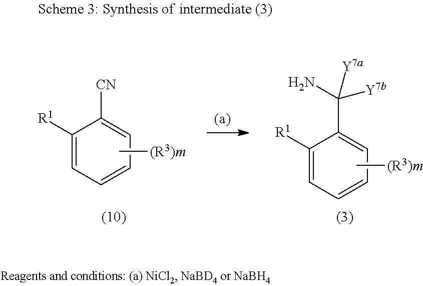

Appropriately deuterated benzyl amine intermediates (3) are produced by treating appropriately deuterated o-tolunitrile (10) with NaBD₄ or NaBD₄ and NiCl₂ in a manner analogous to the procedure described by Khurana, J., et al., Synthetic Communications, 32(8), 1265-1269; 2002. Alternatively, intermediate (10) may be treated with LiAlD₄ or LiAlH₄ to furnish correspondingly deuterated intermediate (3) in a manner analogous to the method described by Armesto, D., et al., Journal of the Chemical Society, Perkin Transactions 1: Organic and Bio-Organic Chemistry (1972-1999), (4), 751-5; 1989.

### Commercially Available:

(10a): R¹=CD₃; m=4 (98 atom % D)
(10b): R¹═CH₃; m=0

Utilising commercially available (10a) in the preparation of intermediate (3), compounds of Formula I can be prepared wherein each deuterium of R¹ has between 95-98% deuterium incorporation and each R³ has between 95-98% deuterium incorporation.

Intermediate (10c), wherein R¹=CD₃ and m=0, can be prepared as outlined in Scheme 3a.

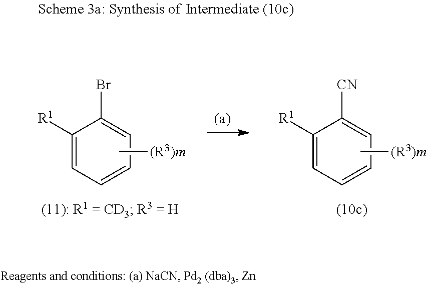

In a manner analogous to the procedure described by Ushkov, A., Journal of the American Chemical Society (2011), 133(28), 10999-11005, Pd-catalyzed cyanation of commercially available 2-(Methyl-d₃)bromobenzene (95 atom % D) (11) using NaCN as cyanide source affords aryl cyanide intermediate (10c). The use of commercially available (11) can thus afford compounds of Formula I, wherein each deuterium of R¹ has between 95-98% deuterium incorporation.

Appropriately deuterated intermediate (5), for use in the preparation of compounds of Formula I according to Scheme 1, may be prepared from corresponding deuterated reagents depicted below.

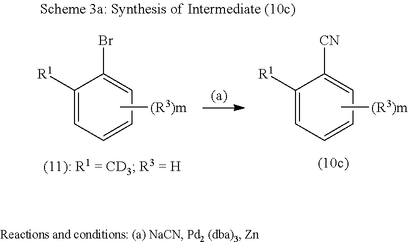

In a manner analogous to a procedure described by Chavan, S., et al., Synthetic Communications, 37(18), 3143-3149; 2007, Michael reaction of appropriately deuterated intermediate (13), wherein Y is hydrogen or deuterium, with appropriately deuterated aryl amine (14) furnishes adduct intermediate (15), wherein each Y is hydrogen or deuterium, which is subsequently submitted to a second round of Michael reaction with intermediate (13′) to afford a double Michael adduct intermediate (16). Appropriately deuterated intermediate (16) is further subjected to Dieckmann condensation using NaH as base to provide appropriately deuterated keto ester (5).

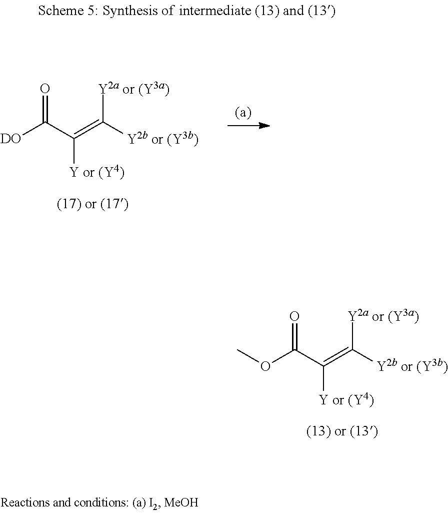

Appropriately deuterated acrylic acid (17)/(17′) is esterified in a manner analogous to a procedure described by Ren, Y., et al. Synthetic Communications, 40(11), 1670-1676; 2010 to produce appropriately deuterated ester intermediate (13). Starting material (17a):acrylic acid-d₄ is commercially available (98 atom % D). Using commercially available (17a) in the preparation of intermediate (13)/(13′), affords compounds of Formula I, wherein each of Y^{2a}, Y^{2b}, Y^{3a} Y^{3b} and Y⁴ has at least 95-98% deuterium incorporation.

The following intermediates (14) are commercially available: Benzyl-d₇-amine (99 atom % D) (14a), benzyl-2,3,4,5,6-d₅-amine (at least 99 atom % D) (14b), and benzyl-alpha,alpha-d₂-amine (98 atom % D) (14c). The use of any one of these commercially available deuterated intermediates, (14), can thus afford compounds of Formula I wherein each Y¹ has at least 95-98% deuterium incorporation.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I, Ia, Ib, Ic and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene, T W et al., Protective Groups in Organic Synthesis, 3^{rd} Ed., John Wiley and Sons (1999); Fieser, L et al., Fieser and Fieser's Reagents for Organic Synthesis, John Wiley and Sons (1994); and Paquette, L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pharmaceutical compositions comprising an effective amount of a compound of Formula I, Ia, Ib or Ic (e.g., including any of the formulae herein), or a pharmaceutically acceptable salt of said compound; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile inj ectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

In some embodiments, the pharmaceutical composition of the invention comprises at least one second therapeutic agent.

In some embodiments, the second therapeutic agent is one or more hormone analogue and/or an antihormone. In one aspect of these embodiments, the hormone analogue and/or an antihormone is selected from tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxy-progesterone, octreotide, and combinations thereof.

In some embodiments, the second therapeutic agent comprises one or more LHRH agonists and/or antagonists. In one aspect of these embodiments, the one or more LHRH agonist is selected from goserelin acetate, leuprolide acetate, triptorelin pamoate and combinations thereof. In another aspect of these embodiments, the one or more LHRH antagonist is selected from the group consisting of Degarelix, Cetrorelix, Abarelix, Ozarelix, Degarelix combinations thereof.

In some embodiments, the second therapeutic agent comprises one or more growth factor inhibitors selected from the group consisting of inhibitors of: platelet derived growth factor (PDGF), fibroblast growth factor (FGF), vascular endothelial growth factor (VEGF), epidermal growth factor (EGF), insulin-like growth factors (IGF), human epidermal growth factor (HER) and hepatocyte growth factor (HGF). In one aspect of these embodiments, the one or more inhibitors of the human epidermal growth factor is selected from the group consisting of HER2, HER3, and HER4.

In some embodiments, the second therapeutic agent comprises one or more tyrosine kinase inhibitors. In one aspect of these embodiments, the tyrosine kinase inhibitor is selected from cetuximab, gefitinib, imatinib, lapatinib and trastuzumab, and combinations thereof. In another aspect of these embodiments, the tyrosine kinase inhibitor is a PTK2/FAK inhibitor.

In some embodiments, the second therapeutic agent comprises one or more aromatase inhibitors. In one aspect of these embodiments, the aromatase inhibitor is selected from anastrozole, letrozole, liarozole, vorozole, exemestane, atamestane, and combinations thereof.

In certain embodiments, the second therapeutic agent comprises one or more antimetabolites which are antifolates. In one aspect of these embodiments, the antifolate is selected from methotrexate, raltitrexed, and pyrimidine analogues.

In certain embodiments, the second therapeutic agent comprises one or more antimetabolites which are pyrimidine analogues. In one aspect of these embodiments, the pyrimidine analogue is selected from the group consisting of 5-fluorouracil, capecitabine and gemcitabine.

In certain embodiments, the second therapeutic agent comprises one or more antimetabolites which are which are purine and/or adenosine analogues. In one aspect of these embodiments, the purine and/or adenosine analogue is selected from mercaptopurine, thioguanine, cladribine and pentostatin, cytarabine, fludarabine, and combinations thereof.

In certain embodiments, the second therapeutic agent comprises one or more antitumour antibiotics. In one aspect of these embodiments, the antitumor antibiotic is selected from anthracyclines, doxorubicin, daunorubicin, epirubicin and idarubicin, mitomycin-C, bleomycin, dactinomycin, plicamycin, streptozocin and combinations thereof.

In certain embodiments, the second therapeutic agent comprises one or more platinum derivatives. In one aspect of these embodiments, the platinum derivative is selected from the group consisting of cisplatin, oxaliplatin, carboplatin and combinations thereof.

In certain embodiments, the second therapeutic agent comprises one or more alkylation agents. In one aspect of these embodiments, the alkylation agent is selected from the group consisting of estramustine, mechlorethamine, melphalan, chlorambucil, busulphan, dacarbazine, cyclophosphamide, ifosfamide, temozolomide, nitrosoureas, and combinations thereof.

In certain embodiments, the second therapeutic agent comprises one or more nitrosoureas. In one aspect of these embodiments, the nitrosourea is selected from the group consisting of carmustine, lomustine, thiotepa, and combinations thereof.

In certain embodiments, the second therapeutic agent comprises one or more comprises antimitotic agents. In one aspect of these embodiments, the antimitotic agent is selected from the group consisting of Vinca alkaloids (e.g., vinblastine, vindesine, vinorelbine, vincristine, and combinations thereof) and taxanes. In a more specific aspect of these embodiments, the second therapeutic agent comprises one or more taxanes selected from the group consisting of paclitaxel, docetaxel, and combinations thereof.

In certain embodiments, the second therapeutic agent comprises one or more topoisomerase inhibitors which are epipodophylotoxins. In one aspect of these embodiments, the epipodophyllotoxin is selected from etoposide and Etopophos, teniposide, amsacrine, topotecan, irinotecan, mitoxantrone, and combinations thereof.

In certain embodiments, the second therapeutic agent comprises one or more serine/threonine kinase inhibitors. In one aspect of these embodiments, the serine/threonine kinase inhibitor is selected from PDK 1 inhibitors, B-Raf inhibitors, mTOR inhibitors, mTORC1 inhibitors, PI3K inhibitors, dual mTOR/PI3K inhibitors, STK 33 inhibitors, AKT inhibitors, PLK 1 inhibitors, inhibitors of CDKs, Aurora kinase inhibitors, and combinations thereof.

In certain embodiments, the second therapeutic agent comprises one or more small molecule multi-kinase inhibitor. In aspect of these embodiments, the small molecule multi-kinase inhibitor is sorafenib or regorafenib.

In certain embodiments, the second therapeutic agent comprises a Hedgehog Pathway Inhibitor. In one aspect of these embodiments, the Hedgehog Pathway Inhibitor is vismodegib.

In certain embodiments, the second therapeutic agent comprises one or more protein interaction inhibitors. In one aspect of these embodiments, the one or more protein interaction inhibitor is selected from IAP, Mcl-1, MDM2/MDMX and combinations thereof.

In certain embodiments, the second therapeutic agent comprises one or more rapamycin analogs. In one aspect of these embodiments, the rapamycin analog is selected from everolimus, temsirolimus, ridaforolimus, sirolimus, and combinations thereof.

In certain embodiments, the second therapeutic agent comprises one or more therapeutic agents selected from amifostin, anagrelid, clodronat, filgrastin, interferon, interferon alpha, leucovorin, rituximab, procarbazine, levamisole, mesna, mitotane, pamidronate and porfimer, and combinations thereof.

In certain embodiments, the second therapeutic agent comprises one or more anti-cancer agent. In one aspect of these embodiments, the anti-cancer agent is selected from acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bevacizumab, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflomithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, fluorocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochlride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, zorubicin and combinations thereof.

In certain embodiments, the second therapeutic agent comprises one or more therapeutic agents selected from 2-chlorodesoxyadenosine, 2-fluorodesoxy-cytidine, 2-methoxyoestradiol, 2C4,3-alethine, 131-1-TM-601, 3CPA, 7-ethyl-10-hydroxycamptothecin, 16-aza-epothilone B, A 105972, A 204197, abiraterone, aldesleukin, alitretinoin, allovectin-7, altretamine, alvocidib, amonafide, anthrapyrazole, AG-2037, AP-5280, apaziquone, apomine, aranose, arglabin, arzoxifene, atamestane, atrasentan, auristatin PE, AVLB, AZ10992, ABX-EGF, AMG-479 (ganitumab), ARRY 162, ARRY 438162, ARRY-300, ARRY-142886/AZD-6244 (selumetinib), ARRY-704/AZD-8330, AR-12, AR-42, AS-703988, AXL-1717, AZD-8055, AZD-5363, AZD-6244, ARQ-736, ARQ 680, AS-703026 (primasertib), avastin, AZD-2014, azacytidine, azaepothilone B, azonafide, BAY-43-9006, BAY 80-6946, BBR-3464, BBR-3576, bevacizumab, BEZ-235, biricodar dicitrate, BCX-1777, BKM-120, bleocin, BLP-25, BMS-184476, BMS-247550, BMS-1 88797, BMS-275291, BMS-663513, BMS-754807, BNP-1350, BNP-7787, BIBW 2992 (afatinib, tomtovok), BIBF 1120 (vargatef), BI 836845, BI 2536, BI 6727, BI 836845, BI 847325, BI 853520, BUB-022, bleomycinic acid, bleomycin A, bleomycin B, brivanib, bryostatin-1, bortezomib, brostallicin, busulphan, BYL-719, CA-4 prodrug, CA-4, CapCell, calcitriol, canertinib, canfosfamide, capecitabine, carboxyphthalatoplatin, CCI-779, CC-115, CC-223, CEP-701, CEP-751, CBT-1 cefixime, ceflatonin, ceftriaxone, celecoxib, celmoleukin, cemadotin, CH4987655/RO-4987655, chlorotrianisene, cilengitide, ciclosporin, CDA-II, CDC-394, CKD-602, CKI-27, clofarabin, coichicin, combretastatin A4, COT inhibitors, CHS-828, CH-5132799, CLL-Thera, CMT-3 cryptophycin 52, CTP-37, CTLA-4 monoclonal antibodies, CP-461, CV-247, cyanomorpholinodoxorubicin, cytarabine, D 24851, decitabine, deoxorubicin, deoxyrubicin, deoxycoformycin, depsipeptide, desoxyepothilone B, dexamethasone, dexrazoxanet, diethylstilbestrol, diflomotecan, didox, DMDC, dolastatin 10, doranidazole, DS-7423, E7010, E-6201, edatrexat, edotreotide, efaproxiral, eflornithine, EGFR inhibitors, EKB-569, EKB-509, enzastaurin, enzalutamide, elsamitrucin, epothilone B, epratuzumab, ER-86526, erlotinib, ET-18-OCH3, ethynylcytidine, ethynyloestradiol, exatecan, exatecan mesylate, exemestane, exisulind, fenretinide, figitumumab, floxuridine, folic acid, FOLFOX, FOLFOX4, FOLFIRI, formestane, fotemustine, galarubicin, gallium maltolate, gefinitib, gemtuzumab, gimatecan, glufosfamide, GCS-100, GDC-0623, GDC-0941 (pictrelisib), GDC-0980, GDC-0032, GDC-0068, GDC-0349, GDC-0879, G17DT immunogen, GMK, GPX-100, gpl00-peptide vaccines, GSK-5126766, GSK-690693, GSK-1120212 (trametinib), GSK-2118436 (dabrafenib), GSK-2126458, GSK-2132231 A, GSK-2334470, GSK-2110183, GSK-2141795, GW2016, granisetron, herceptine, hexamethylmelamine, histamine, homoharringtonine, hyaluronic acid, hydroxyurea, hydroxyprogesterone caproate, ibandronate, ibrutinib, ibritumomab, idatrexate, idenestrol, IDN-5109, IGF-1R inhibitors, IMC-1C11, IMC-A12 (cixutumumab), immunol, indisulam, interferon alpha-2a, interferon alpha-2b, pegY^{1a} ted interferon alpha-2b, interleukin-2, INK-1117, INK-128, INSM-18, ionafarnib, ipilimumab, iproplatin, irofulven, isohomohalichondrin-B, isoflavone, isotretinoin, ixabepilone, JRX-2, JSF-154, J-107088, conjugated oestrogens, kahalid F, ketoconazole, KW-2170, KW-2450, lobaplatin, leflunomide, lenograstim, leuprolide, leuporelin, lexidronam, LGD-1550, linezolid, lutetium texaphyrin, lometrexol, losoxantrone, LU 223651, lurtotecan, LY-S6AKT1, LY-2780301, mafosfamide, marimastat, mechloroethamine, MEK inhibitors, MEK-162, methyltestosteron, methylprednisolone, MEDI-573, MEN-10755, MDX-H210, MDX-447, MDX-1379, MGV, midostaurin, minodronic acid, mitomycin, mivobulin, MK-2206, MK-0646 (dalotuzumab), MLN518, motexaf in gadolinium, MS-209, MS-275, MX6, neridronate, neratinib, Nexavar, neovastat, nilotinib, nimesulide, nitroglycerin, nolatrexed, norelin, N-acetylcysteine, 06-benzylguanine, oblimersen, omeprazole, oncophage, oncoVEXGM-CSF, ormiplatin, ortataxel, OX44 antibodies, OSI-027, OSI-906 (linsitinib), 4-1BB antibodies, oxantrazole, oestrogen, panitumumab, patupilone, pegfilgrastim, PCK-3145, pegfilgrastim, PBI-1402, PBI-05204, PD0325901, PD-1 antibodies, PEG-paclitaxel, albumin-stabilized paclitaxel, PEP-005, PF-05197281, PF-05212384, PF-04691502, PHT-427, P-04, PKC412, P54, PI-88, pelitinib, pemetrexed, pentrix, perifosine, perillylalcohol, pertuzumab, PI3K inhibitors, PI3K/mTOR inhibitors, PG-TXL, PG2, PLX-4032/RO-5185426 (vemurafenib), PLX-3603/RO-5212054, PT-100, PWT-33597, PX-866, picoplatin, pivaloyloxymethylbutyrate, pixantrone, phenoxodiol O, PKI166, plevitrexed, plicamycin, polyprenic acid, porfiromycin, prednisone, prednisolone, quinamed, quinupristin, R115777, RAF-265, ramosetron, ranpirnase, RDEA-119/BAY 869766, RDEA-436, rebeccamycin analogues, receptor tyrosine kinase (RTK) inhibitors, revimid, RG-7167, RG-7304, RG-7421, RG-7321, RG 7440, rhizoxin, rhu-MAb, rinfabate, risedronate, rituximab, robatumumab, rofecoxib, RO-31-7453, RO-5126766, RO-5068760, RPR 109881A, rubidazone, rubitecan, R-flurbiprofen, RX-0201, S-9788, sabarubicin, SAHA, sargramostim, satraplatin, SB 408075, Se-015/Ve-015, SU5416, SU6668, SDX-101, semustin, seocalcitol, SM-11355, SN-38, SN-4071, SR-27897, SR-31747, SR-13668, SRL-172, sorafenib, spiroplatin, squalamine, suberanilohydroxamic acid, sutent, T 900607, T 138067, TAK-733, TAS-103, tacedinaline, talaporfin, Tarceva, tariquitar, tasisulam, taxotere, taxoprexin, tazarotene, tegafur, temozolamide, tesmilifene, testosterone, testosterone propionate, tesmilifene, tetraplatin, tetrodotoxin, tezacitabine, thalidomide, theralux, therarubicin, thymalfasin, thymectacin, tiazofurin, tipifarnib, tirapazamine, tocladesine, tomudex, toremofin, trabectedin, TransMID-107, transretinic acid, traszutumab, tremelimumab, tretinoin, triacetyluridine, triapine, triciribine, trimetrexate, TLK-286TXD 258, tykerb/tyverb, urocidin, valrubicin, vatalanib, vincristine, vinflunine, virulizin, WX-UK1, WX-554, vectibix, xeloda, XELOX, XL-147, XL-228, XL-281, XL-518/R-7420/GDC-0973, XL-765, YM-511, YM-598, ZD-4190, ZD-6474, ZD-4054, ZD-0473, ZD-6126, ZD-9331, ZD1839, ZSTK-474, zoledronat, zosuquidar, and combinations thereof.

In certain embodiments, the second therapeutic agent is selected from Allopurinol, Arsenic Trioxide, Azacitidine, Bortezomib, Bevacizumab, Capecitabine, Carboplatin, Celecoxib, Chlorambucil, Clofarabine, Cytarabine, Dacarbazine, Daunorubicin HCl, Docetaxel, Doxorubicin HCl, Floxuridine, Gemcitabine HCl, Hydroxyurea, Ifosfamide, Imatinib Mesylate, Ixabepilone, Lenalidomide, Megestrol acetate, Methotrexate, Mitotane, Mitoxantrone HCl, Oxaliplatin, Paclitaxel, Pralatrexate, Romidepsin, Sorafenib, Streptozocin, Tamoxifen Citrate, Topotecan HCl, Tretinoin, Vandetanib, Vismodegib, Vorinostat, and combinations thereof.

In some embodiments, the second therapeutic agent comprises another drug that target tumor necrosis factor-related apoptosis-inducing ligand (TRAIL) receptors. In one aspect of these embodiments, the second therapeutic agent is a recombinant TRAIL or an agonistic antibody that activates one or more TRAIL receptors. In an alternate aspect of these embodiments, the second therapeutic agent includes one or more antibodies or recombinant TRAIL that activate signaling by DR4 and/or DR5. In a more specific aspect of these embodiments, the second therapeutic agent includes one or more of mapatumumab, lexatumumab, Apomab, AMG-655, LBY-135 and rhApo2L/TRAIL.

In other embodiments, the second therapeutic agent includes an active agent selected from Camptothecin, 5-FU, capecitabine, cisplatin, doxorubicin, irinotecan, paclitaxel, cisplatin, bortezomib, BH3I-2, rituximab, radiation, triterpenoids, sorafenib, gemcitabine, HDAC inhibitors, carboplatin, T-101 (a gossypol derivate), ABT-263, ABT-737, and GX-15-070 (obatoclax), vorinostat, cetuximab, panitumumab, bevacizumab, ganitumab, interferon gamma, sorafenib, XIAP antagonists, Bcl-2 antagonists, and Smac mimetics.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat the target disorder.

The interrelationship of dosages for animals and humans (based on milligrams per meter squared of body surface) is described in Freireich et al., Cancer Chemother. Rep, 1966, 50: 219. Body surface area may be approximately determined from height and weight of the subject. See, e.g., Scientific Tables, Geigy Pharmaceuticals, Ardsley, N.Y., 1970, 537.

In one embodiment, an effective amount of a compound of this invention can ranges from between about 10 to 2000 mg/dose. In one aspect of this embodiment, an effective dose of a compound of this invention ranges from about 20 to 1000 mg/dose. In another aspect of this embodiment, an effective dose of a compound of this invention ranges from about 20 to 500 mg/dose. In another aspect of this embodiment, an effective dose of a compound of this invention ranges from about 20 to 250 mg/dose. In another aspect of this embodiment, an effective dose of a compound of this invention ranges from about 20 to 100 mg/dose. In another aspect of this embodiment, an effective dose of a compound of this invention ranges from about 100 to 500 mg/dose. In another aspect of this embodiment, an effective dose of a compound of this invention ranges from about 100 to 250 mg/dose. The compound of this invention may be administered from one to four times/month to achieve the effective amount. In a more specific aspect, the compound of this invention is administered one to two times/month to achieve the effective amount.

In an alternate embodiment, an effective amount of a compound of this invention can range from between about 0.1 mg/kg to about 40 mg/kg.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In one embodiment, the invention provides a method of modulating the activity of TRAIL in a cell, comprising contacting a cell with one or more compounds of Formula I, Ia, Ib or Ic herein, or a pharmaceutically acceptable salt thereof.

According to another embodiment, the invention provides a method of treating a cancer in a subject, comprising the step of administering to the subject an effective amount of a compound or a composition of this invention. In one embodiment the subject is a patient in need of such treatment.

In one embodiment the cancer is selected from the group consisting of acute lymphoblastic leukemia, acute myeloid leukemia, adrenocortical carcinoma, AIDS-related cancers, AIDS-related lymphoma, anal or rectal cancer, appendix cancer, astrocytomas, and atypical teratoid/rhabdoid tumor.

In one embodiment, the cancer is selected from the group consisting of basal cell carcinoma, bile duct cancer, bladder cancer, bone cancer, osteosarcoma and malignant fibrous histiocytoma, brain tumor, breast cancer, bronchial tumors, Burkitt Lymphoma, and spinal cord tumors.

In one embodiment, the cancer is selected from the group consisting of Carcinoid Tumor, Carcinoma of Unknown Primary, Central Nervous System Atypical Teratoid/Rhabdoid Tumor, Central Nervous System Embryonal Tumors, Central Nervous System Lymphoma, Cervical Cancer, Chordoma, Chronic Lymphocytic Leukemia, Chronic Myelogenous Leukemia, Chronic Myeloproliferative Disorders, Colon Cancer, Colorectal Cancer, Craniopharyngioma, and Cutaneous T-Cell Lymphoma.

In one embodiment, the cancer is selected from the group consisting of Embryonal Tumors of Central Nervous System, Endometrial Cancer, Ependymoblastoma, Ependymoma, Esophageal Cancer, Ewing Sarcoma Family of Tumors, Extracranial Germ Cell Tumor, Extragonadal Germ Cell Tumor, Extrahepatic Bile Duct Cancer, and Eye Cancer.

In one embodiment, the cancer is selected from Gallbladder Cancer, Gastric (Stomach) Cancer, Gastrointestinal Carcinoid Tumor, Gastrointestinal Stromal Tumor (GIST), Germ Cell Tumor, Gestational Trophoblastic Tumor, and Glioma.

In one embodiment, the cancer is selected from the group consisting of Hairy Cell Leukemia, Head and Neck Cancer, Hepatocellular (Liver) Cancer, Histiocytosis, Hodgkin Lymphoma, and Hypopharyngeal Cancer.

In one embodiment, the cancer is selected from the group consisting of Kaposi Sarcoma, and Kidney (Renal Cell) Cancer.

In one embodiment, the cancer is selected from the group consisting of Langerhans Cell Histiocytosis, Laryngeal Cancer, Lip and Oral Cavity Cancer, Liver Cancer, Lung Cancer, Non-Hodgkin Lymphoma, and Primary Central Nervous System Lymphoma.

In one embodiment, the cancer is selected from the group consisting of Waldenstrom's macroglobulinemia (lymphoplasmacytic lymphoma), Malignant Fibrous Histiocytoma of Bone and Osteosarcoma, Medulloblastoma, Medulloepithelioma, Melanoma, Merkel Cell Carcinoma, Mesothelioma, Metastatic Squamous Neck Cancer with Occult Primary, Multiple Endocrine Neoplasia Syndrome, Mouth Cancer, Multiple Myeloma/Plasma Cell Neoplasm, Mycosis Fungoides, Myelodysplastic Syndromes, Myelodysplastic/Myeloproliferative Neoplasms, Multiple Myeloma, and Myeloproliferative Disorders.

In one embodiment, the cancer is selected from the group consisting of Nasal Cavity and Paranasal Sinus Cancer, Nasopharyngeal Cancer, and Neuroblastoma.

In one embodiment, the cancer is selected from the group consisting of Oral Cancer, Lip and Oral Cavity Cancer, Oropharyngeal Cancer, Osteosarcoma and Malignant Fibrous Histiocytoma of Bone, Ovarian Cancer, Ovarian Germ Cell Tumor, Ovarian Epithelial Cancer, and Ovarian Low Malignant Potential Tumor.

In one embodiment, the cancer is selected from the group consisting of Pancreatic Cancer, Papillomatosis, Paranasal Sinus and Nasal Cavity Cancer, Parathyroid Cancer, Penile Cancer, Pharyngeal Cancer, Pineal Parenchymal Tumors of Intermediate Differentiation, Pineoblastoma and Supratentorial Primitive Neuroectodermal Tumors, Pituitary Tumor, Pleuropulmonary Blastoma, Pregnancy and Breast Cancer, Primary Central Nervous System Lymphoma, and Prostate Cancer.

In one embodiment, the cancer is selected from the group consisting of Rectal Cancer, Renal Cell (Kidney) Cancer, Renal Pelvis and Ureter, Respiratory Tract Carcinoma Involving the NUT Gene on Chromosome 15, Retinoblastoma, and Rhabdomyosarcoma.

In one particular embodiment, the method of this invention is used to treat a cancer selected from glioblastoma, colorectal cancer, breast cancer (e.g., triple-negative breast cancer, non-small cell lung cancer, and other solid tumors in a subject in need thereof.

Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to the subject in need thereof one or more second therapeutic agents. The choice of second therapeutic agent is also dependent upon the particular disease or condition to be treated. Examples of second therapeutic agents that may be employed in the methods of this invention are those set forth above for use in combination compositions comprising a compound of this invention and one or more second therapeutic agents.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described second therapeutic agents in the manufacture of a medicament, either as a single composition or as separate dosage forms, for treatment in a subject of a disease, disorder or symptom set forth above. Another aspect of the invention is a compound of Formula I, Ia, Ib or Ic for use in the treatment in a subject of a disease, disorder or symptom thereof delineated herein.

### Example X. Evaluation of Metabolic Stability

Microsomal Assay:

Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability:

7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated counterpart of the compound of Formula I and the positive control, 7-ethoxycoumarin (1 μM). Testing is done in triplicate.

Data Analysis:

The in vitro t_{1/2}s for test compounds are calculated from the slopes of the linear regression of % parent remaining (ln) vs incubation time relationship.

in vitro t=0.693/k

k=−[slope of linear regression of % parent remaining (ln) vs incubation time]

Data analysis is performed using Microsoft Excel Software.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of Formula I: 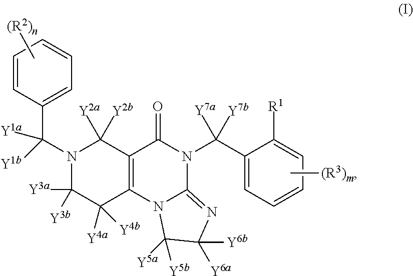
or a pharmaceutically acceptable salt thereof, wherein:
each of Y^{1a}, Y^{1b}, Y^{2a}, Y^{2b}, Y^{3a}, Y^{3b}, Y^{4a}, Y^{4b}, Y^{5a} Y^{5b}, Y^{6a}, Y^{6b}, Y^{7a}, and Y^{7b} are independently selected from hydrogen and deuterium;
R¹ is —CH₃, or —CD₃;
each R², when present, is deuterium;
each R³, when present, is deuterium;
n is 0, 1, 2, 3, 4, or 5;
m is 0, 1, 2, 3, or 4; and
when R¹ is —CH₃, n is 0 and m is 0, then at least one of Y^{1a}, Y^{1b} Y^{2a}, Y^{2b}, Y^{3a}, Y^{3b}, Y^{4a}, Y^{4b}, Y^{5a}, Y^{5b}, Y^{6a}, Y^{6b}, Y^{7a}, and Y^{7b} is deuterium.

2. The compound of claim 1, wherein Y^{1a} and Y^{1b} are the same; Y^{2a} and Y^{2b} are the same; Y^{3a} and Y^{3b} are the same; Y^{4a} and Y^{4b} are the same; Y^{5a} and Y^{5b} are the same; Y^{6a} and Y^{6b} are the same; and Y^{7a} and Y^{7b} are the same.

3. The compound of claim 2, wherein Y^{2a}, Y^{2b}, Y^{3a}, Y^{3b}, Y^{4a} and Y^{4b} are the same; and Y^{5a}, Y^{5b}, Y^{6a} and Y^{6b} are the same.

4. (canceled)

5. The compound of claim 1, wherein the compound of Formula I is a compound of Formula Ia: 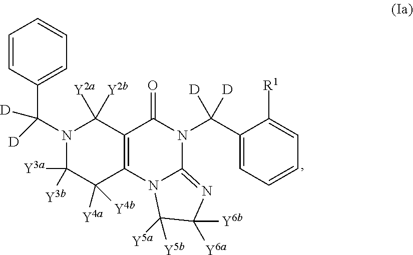
or a pharmaceutically acceptable salt thereof, wherein:
Y^{2a} and Y^{2b} are the same; Y^{3a} and Y^{3b} are the same; Y^{4a} and Y^{4b} are the same; Y^{5a} and Y^{5b} are the same; and Y^{6a} and Y^{6b} are the same; and
R¹ is selected from —CH₃, and —CD₃.

6. The compound of claim 1, wherein the compound of Formula I is a compound of Formula Ib: 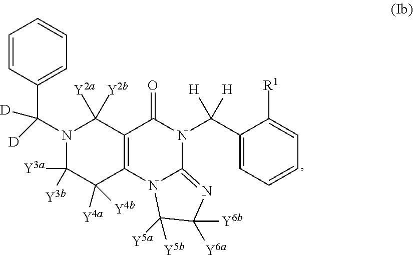
or a pharmaceutically acceptable salt thereof, wherein:
Y^{2a} and Y^{2b} are the same; Y^{3a} and Y^{3b} are the same; Y^{4a} and Y^{4b} are the same; Y^{5a} and Y^{5b} are the same; and Y^{6a} and Y^{6b} are the same; and
R¹ is selected from —CH₃, and —CD₃.

7. The compound of claim 1, wherein the compound of Formula I is a compound of Formula Ic: 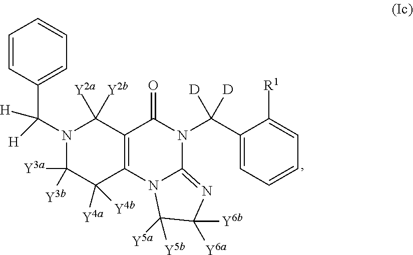
or a pharmaceutically acceptable salt thereof, wherein:
Y^{2a} and Y^{2b} are the same; Y^{3a} and Y^{3b} are the same; Y^{4a} and Y^{4b} are the same; Y^{5a} and Y^{5b} are the same; and Y^{6a} and Y^{6b} are the same; and
R¹ is selected from —CH₃, and —CD₃.

8. (canceled)

9. The compound of claim 1, wherein R¹ is —CD₃.

10. The compound of claim 1, wherein R¹ is —CH₃.

11-15. (canceled)

16. The compound of claim 1, wherein n is 0 or 5, and m is 0 or 4.

17. (canceled)

18. The compound of claim 1, wherein any atom not designated as deuterium is present at its natural isotopic abundance.

19-20. (canceled)

21. The compound of claim 5, wherein R¹ is —CD₃; and the compound is selected from any one of the compounds set forth below:

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 200a | H | H | H | H | H |
| 201a | D | H | H | H | H |
| 202a | H | D | H | H | H |
| 203a | H | H | D | H | H |
| 204a | H | H | H | D | H |
| 205a | H | H | H | H | D |
| 206a | D | D | H | H | H |
| 207a | D | H | D | H | H |
| 208a | D | H | H | D | H |
| 209a | D | H | H | H | D |
| 210a | H | D | D | H | H |
| 211a | H | D | H | D | H |
| 212a | H | D | H | H | D |
| 213a | H | H | D | D | H |
| 214a | H | H | D | H | D |
| 215a | H | H | H | D | D |
| 216a | D | D | D | H | H |
| 217a | D | D | H | D | H |
| 218a | D | D | H | H | D |
| 219a | D | H | D | D | H |
| 220a | D | H | D | H | D |
| 221a | D | H | H | D | D |
| 222a | H | D | D | D | H |
| 223a | H | D | D | H | D |
| 224a | H | D | H | D | D |
| 225a | H | H | D | D | D |
| 226a | D | D | D | D | H |
| 227a | D | D | D | H | D |
| 228a | D | D | H | D | D |
| 229a | D | H | D | D | D |
| 230a | H | D | D | D | D |
| 231a | D | D | D | D | D |

or a pharmaceutically acceptable salt thereof, wherein any atom not designated as deuterium is present at its natural isotopic abundance.

22. The compound of claim 5, wherein R¹ is —CH₃; and the compound is selected from any one of the compounds set forth below:

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 200b | H | H | H | H | H |
| 201b | D | H | H | H | H |
| 202b | H | D | H | H | H |
| 203b | H | H | D | H | H |
| 204b | H | H | H | D | H |
| 205b | H | H | H | H | D |
| 206b | D | D | H | H | H |
| 207b | D | H | D | H | H |
| 208b | D | H | H | D | H |
| 209b | D | H | H | H | D |
| 210b | H | D | D | H | H |
| 211b | H | D | H | D | H |
| 212b | H | D | H | H | D |
| 213b | H | H | D | D | H |
| 214b | H | H | D | H | D |
| 215b | H | H | H | D | D |
| 216b | D | D | D | H | H |
| 217b | D | D | H | D | H |
| 218b | D | D | H | H | D |
| 219b | D | H | D | D | H |
| 220b | D | H | D | H | D |
| 221b | D | H | H | D | D |
| 222b | H | D | D | D | H |
| 223b | H | D | D | H | D |
| 224b | H | D | H | D | D |
| 225b | H | H | D | D | D |
| 226b | D | D | D | D | H |
| 227b | D | D | D | H | D |
| 228b | D | D | H | D | D |
| 229b | D | H | D | D | D |
| 230b | H | D | D | D | D |
| 231b | D | D | D | D | D |

or a pharmaceutically acceptable salt thereof, wherein any atom not designated as deuterium is present at its natural isotopic abundance.

23. The compound of claim 6, wherein R¹ is —CD₃; and the compound is selected from any one of the compounds set forth below:

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 300a | H | H | H | H | H |
| 301a | D | H | H | H | H |
| 302a | H | D | H | H | H |
| 303a | H | H | D | H | H |
| 304a | H | H | H | D | H |
| 305a | H | H | H | H | D |
| 306a | D | D | H | H | H |
| 307a | D | H | D | H | H |
| 308a | D | H | H | D | H |
| 309a | D | H | H | H | D |
| 310a | H | D | D | H | H |
| 311a | H | D | H | D | H |
| 312a | H | D | H | H | D |
| 313a | H | H | D | D | H |
| 314a | H | H | D | H | D |
| 315a | H | H | H | D | D |
| 316a | D | D | D | H | H |
| 317a | D | D | H | D | H |
| 318a | D | D | H | H | D |
| 319a | D | H | D | D | H |
| 320a | D | H | D | H | D |
| 321a | D | H | H | D | D |
| 322a | H | D | D | D | H |
| 323a | H | D | D | H | D |
| 324a | H | D | H | D | D |
| 325a | H | H | D | D | D |
| 326a | D | D | D | D | H |
| 327a | D | D | D | H | D |
| 328a | D | D | H | D | D |
| 329a | D | H | D | D | D |
| 330a | H | D | D | D | D |
| 331a | D | D | D | D | D |

or a pharmaceutically acceptable salt thereof, wherein any atom not designated as deuterium is present at its natural isotopic abundance.

24. The compound of claim 6, wherein R¹ is —CH₃; and the compound is selected from any one of the compounds set forth below:

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 300b | H | H | H | H | H |
| 301b | D | H | H | H | H |
| 302b | H | D | H | H | H |
| 303b | H | H | D | H | H |
| 304b | H | H | H | D | H |
| 305b | H | H | H | H | D |
| 306b | D | D | H | H | H |
| 307b | D | H | D | H | H |
| 308b | D | H | H | D | H |
| 309b | D | H | H | H | D |
| 310b | H | D | D | H | H |
| 311b | H | D | H | D | H |
| 312b | H | D | H | H | D |
| 313b | H | H | D | D | H |
| 314b | H | H | D | H | D |
| 315b | H | H | H | D | D |
| 316b | D | D | D | H | H |
| 317b | D | D | H | D | H |
| 318b | D | D | H | H | D |
| 319b | D | H | D | D | H |
| 320b | D | H | D | H | D |
| 321b | D | H | H | D | D |
| 322b | H | D | D | D | H |
| 323b | H | D | D | H | D |
| 324b | H | D | H | D | D |
| 325b | H | H | D | D | D |
| 326b | D | D | D | D | H |
| 327b | D | D | D | H | D |
| 328b | D | D | H | D | D |
| 329b | D | H | D | D | D |
| 330b | H | D | D | D | D |
| 331b | D | D | D | D | D |

or a pharmaceutically acceptable salt thereof, wherein any atom not designated as deuterium is present at its natural isotopic abundance.

25. The compound of claim 7, wherein R¹ is —CD₃; and the compound is selected from any one of the compounds set forth below:

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 400a | H | H | H | H | H |
| 401a | D | H | H | H | H |
| 402a | H | D | H | H | H |
| 403a | H | H | D | H | H |
| 404a | H | H | H | D | H |
| 405a | H | H | H | H | D |
| 406a | D | D | H | H | H |
| 407a | D | H | D | H | H |
| 408a | D | H | H | D | H |
| 409a | D | H | H | H | D |
| 410a | H | D | D | H | H |
| 411a | H | D | H | D | H |
| 412a | H | D | H | H | D |
| 413a | H | H | D | D | H |
| 414a | H | H | D | H | D |
| 415a | H | H | H | D | D |
| 416a | D | D | D | H | H |
| 417a | D | D | H | D | H |
| 418a | D | D | H | H | D |
| 419a | D | H | D | D | H |
| 420a | D | H | D | H | D |
| 421a | D | H | H | D | D |
| 422a | H | D | D | D | H |
| 423a | H | D | D | H | D |
| 424a | H | D | H | D | D |
| 425a | H | H | D | D | D |
| 426a | D | D | D | D | H |
| 427a | D | D | D | H | D |
| 428a | D | D | H | D | D |
| 429a | D | H | D | D | D |
| 430a | H | D | D | D | D |
| 431a | D | D | D | D | D |

or a pharmaceutically acceptable salt thereof, wherein any atom not designated as deuterium is present at its natural isotopic abundance.

26. The compound of claim 7, wherein R¹ is —CH₃; and the compound is selected from any one of the compounds set forth below:

| Cmpd | | | | | |
|---|---|---|---|---|---|
| No. | Y^{2a}/Y^{2b} | Y^{3a}/Y^{3b} | Y^{4a}/Y^{4b} | Y^{5a}/Y^{5b} | Y^{6a}/Y^{6b} |
| 400b | H | H | H | H | H |
| 401b | D | H | H | H | H |
| 402b | H | D | H | H | H |
| 403b | H | H | D | H | H |
| 404b | H | H | H | D | H |
| 405b | H | H | H | H | D |
| 406b | D | D | H | H | H |
| 407b | D | H | D | H | H |
| 408b | D | H | H | D | H |
| 409b | D | H | H | H | D |
| 410b | H | D | D | H | H |
| 411b | H | D | H | D | H |
| 412b | H | D | H | H | D |
| 413b | H | H | D | D | H |
| 414b | H | H | D | H | D |
| 415b | H | H | H | D | D |
| 416b | D | D | D | H | H |
| 417b | D | D | H | D | H |
| 418b | D | D | H | H | D |
| 419b | D | H | D | D | H |
| 420b | D | H | D | H | D |
| 421b | D | H | H | D | D |
| 422b | H | D | D | D | H |
| 423b | H | D | D | H | D |
| 424b | H | D | H | D | D |
| 425b | H | H | D | D | D |
| 426b | D | D | D | D | H |
| 427b | D | D | D | H | D |
| 428b | D | D | H | D | D |
| 429b | D | H | D | D | D |
| 430b | H | D | D | D | D |
| 431b | D | D | D | D | D |

or a pharmaceutically acceptable salt thereof, wherein any atom not designated as deuterium is present at its natural isotopic abundance.

27. The compound of claim 1, wherein each position that is designated as deuterium has at least 80%, at least 85%, at least 90%, at least 95%, at least 97% or at least 99% deuterium incorporation at that position.

28. A pharmaceutical composition comprising a compound of claim 1; and a pharmaceutically acceptable carrier.

29. A method of inducing tumor necrosis factor (TNF) related apoptosis-inducing ligand (TRAIL) in a cell, comprising the step of contacting the cell with a compound of claim 1.

30. A method of treating a cancer selected from glioblastoma, colorectal cancer, breast cancer, non-small cell lung cancer, and solid tumors, comprising the step of administering to a subject in need thereof a compound of claim 1.

31. The compound of claim 27, wherein each position that is designated as deuterium has at least 90% deuterium incorporation at that position.

32. The compound of claim 27, wherein each position that is designated as deuterium has at least 95% deuterium incorporation at that position.
